(19) United States
(12) Patent Application Publication
(10) Pub. No.: US 2026/0306524 A1
(43) Pub. Date: Oct. 8, 2026

(54) Guanidinylated Amphiphilic Tobramycin Derivates and B-Lactam/B-Lactamase Inhibitor Combinations for Treatment of Pseudomonas Aeruginosa Infections

(71) Applicant: University of Manitoba, Winnipeg (CA)

(72) Inventors: Frank Schweizer, Winnipeg (CA); Danzel Ramirez, Winnipeg (CA)

(21) Appl. No.: 19/478,043

(22) PCT Filed: May 9, 2024
(86) PCT No.: PCT/CA2024/050626

§ 371 Date: Oct. 23, 2025

Related U.S. Application Data
(60) Provisional application No. 63/501,837, filed on May 12, 2023.

(51) Int. Cl. A61K 31/7036; A61K 31/407; A61K 31/427; A61K 31/496; A61K 31/546; A61K 31/551; A61P 31/04

(52) U.S. Cl. CPC A61K 31/7036; A61K 31/407; A61K 31/427; A61K 31/496; A61K 31/546; A61K 31/551; A61P 31/04

(57) ABSTRACT

The effectiveness of β-lactam antibiotics when used in conjunction with a β-lactamase inhibitor to treat Pseudomonas aeruginosa infections is enhanced with the inclusion of an outer membrane permeabilizing tobramycin derivative. The optimized guanidinylated tobramycin-biphenyl derivatives are non-cytotoxic and demonstrated the ability to potentiate ceftazidime and aztreonam monotherapy and in dual combinations with avibactam against multidrug-resistant and f-lactamase harboring isolates of P. aeruginosa. The triple combination of ceftazidime/avibactam plus guanidinylated tobramycin-biphenyl resulted in a rapid bactericidal activity within 4-8 hours of treatment, demonstrating the potential application of these guanidinylated amphiphilic tobramycin derivatives in augmenting BL/BLI.

### PRIOR APPLICATION INFORMATION

The instant application claims the benefit of US Provisional Patent Application U.S. Ser. No. 63/501,837, filed May 12, 2023 and entitled “GUANIDINYLATED AMPHIPHILIC TOBRAMYCIN DERIVATIVES AND B-LACTAM/B-LACTAMASE INHIBITOR COMBINATIONS FOR TREATMENT OF PSEUDOMONAS AERUGINOSA INFECTIONS”, the entire content of which is hereby incorporated herein by reference for all purposes.

### BACKGROUND OF THE INVENTION

The accelerating emergence of antimicrobial resistance, which is predicted to have a fatal impact on global health, necessitates the development of new and effective treatment options (1). In a list issued by the World Health Organization (WHO) to address this matter and provide guidance for research and development, carbapenem-resistant Pseudomonas aeruginosa was classified as one of the critical priority pathogens (1). Based on recent statistical studies, multidrug-resistant (MDR) P. aeruginosa is becoming a universal concern, particularly in health care settings (2). It is one of the main causes of hospital-acquired infections, capable of causing opportunistic infections in immunocompromised patients and forming highly physically and chemically-resilient biofilm on medical equipment (2-4).

In order to preserve the efficacy of antipseudomonal drugs, the adjuvant strategy has been employed to target various resistance mechanisms. Adjuvants or potentiators are molecules that improve the potency of a primary antibiotic (5-7). For instance, β-lactam (BL) antibiotics are combined with β-lactamase inhibitors (BLI) to prevent hydrolysis by β-lactamase enzymes and have been widely used for the treatment of infections caused by Gram-negative bacteria (8, 9). Among the recently approved BL/BLI combinations that are effective against MDR P. aeruginosa are meropenem/vaborbactam (8), ceftazidime/avibactam (8), ceftolozane/tazobactam (8, 9) and imipenem/relebactam (9). While none of these combinations are effective against metallo-β-lactamase (MBL) producing P. aeruginosa, aztreonam/avibactam is currently in phase III clinical trials and presents itself as a viable treatment option (10, 11).

The addition of an aminoglycoside in BL/BLI combinations has also been recommended as therapy for P. aeruginosa infections (12). Amikacin has been extensively reported to synergize or display in vitro activity with various BL/BLI combinations such as piperacillin/tazobactam (13), ceftolozane/tazobactam (14, 15) and imipenem/relebactam (16). Studies have also displayed the effectiveness of tobramycin with piperacillin/tazobactam (17) and in vitro synergy with ceftazidime/avibactam (18). The synergistic interaction is presumed to arise from the ability of the aminoglycosides to disrupt the outer membrane (OM) and enhance the periplasmic concentration of the BL antibiotic (19, 20). The inclusion of an OM permeabilizing adjuvant based on membrane-acting agents such as aminoglycosides (21-23) and polymyxins (24) has also been investigated. OM permeabilizers in the form of tobramycin conjugates have been reported to synergize with BL antibiotics such as piperacillin, aztreonam, cephalosporins and carbapenems (21, 23, 25, 26). These hybrid molecules consist of a secondary domain linked to tobramycin via an alkyl tether and lack ribosomal binding activity with the C-5 position used as a point of attachment. Despite the lack of antibacterial activity, these adjuvants utilize the “self-promoted uptake” mechanism (27, 28) of the parent compound to induce membrane destabilizing effects. The mechanism is characterized by the displacement of membrane stabilizing divalent magnesium and calcium cations, which initially bridge lipid A phosphates in the lipopolysaccharide (LPS) leaflet of the OM (27, 28). The polycationic nature of these adjuvants allow for the electrostatic interactions with the negatively charged phosphate groups and resulting in the removal of these cations (27, 28). In addition, the lipophilic segment is postulated contribute to OM expansion by inserting into the LPS layer, as demonstrated by amphiphilic neamine derivatives (29).

### SUMMARY OF THE INVENTION

According to an aspect of the invention, there is provided a method of treating a Pseudomonas aeruginosa infection comprising:
administering an effective amount of a compound having a formula as set forth below for compound 5 or compound 8 to an individual who has or is suspected of having a Pseudomonas aeruginosa infection

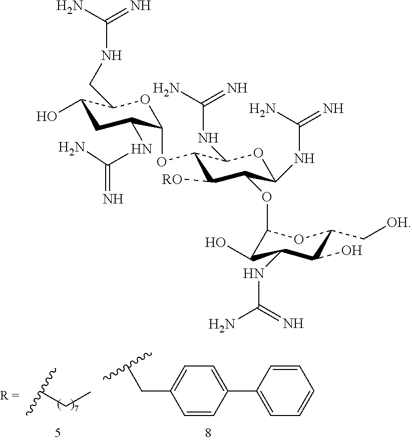

According to another aspect of the invention, there is provided a method of permeabilizing a Gram Negative bacteria outer membrane comprising:
administering an effective amount of a compound having a formula as set forth below for compound 5 or compound 8 to a Gram Negative bacteria

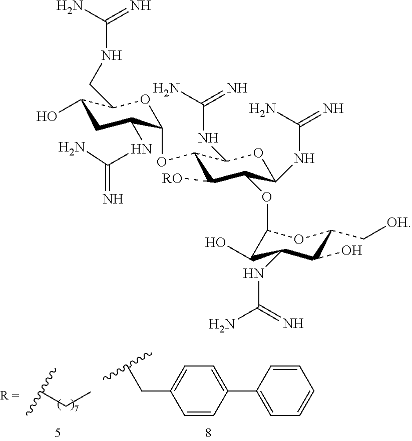

According to another aspect of the invention, there is provided a compound having a formula as set forth below for compound 5 or compound 8 for permeabilizing a Gram Negative bacteria outer membrane

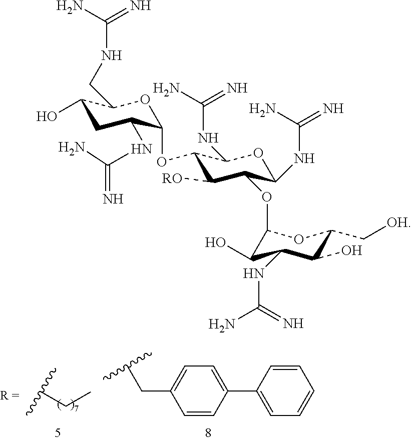

### BRIEF DESCRIPTION OF THE DRAWINGS

FIG. 1. Structures of tobramycin derivatives 2-8 used in this study.

FIG. 2. Fold potentiation of BL antibiotics at 8 μg/mL of adjuvant and at 0.25 μg/mL of tobramycin against P. aeruginosa PAO1.

FIG. 3. MIC of ceftazidime monotherapy and in combination with 8 μg/mL of 5 and 8 against clinical isolates of P. aeruginosa. Dashed line indicates susceptibility breakpoint of ceftazidime.

FIG. 4. MIC of aztreonam monotherapy and in combination with 8 μg/mL of 5 and 8 against clinical isolates of P. aeruginosa. Dashed line indicates susceptibility breakpoint of aztreonam.

FIG. 5. Dual and triple combinations of ceftazidime with avibactam and compounds 5 and 8 against β-lactamase harboring P. aeruginosa. Dashed line indicates susceptibility breakpoint of ceftazidime.

FIG. 6. Dual and triple combinations of aztreonam with avibactam and compounds 5 and 8 against β-lactamase harboring P. aeruginosa. Dashed line indicates susceptibility breakpoint of aztreonam.

FIG. 7. Time-kill curves of ceftazidime monotherapy, dual combination with avibactam and 8 and triple combination with avibactam and 8 against (a) PA 86052 and (b) PA 107092.

FIG. 8. Measurement of OM permeabilization via NPN uptake induced by compounds 5 and 8 with PMBN as control in P. aeruginosa PAO1 cells.

FIG. 9. Cytotoxicity of tobramycin derivarives against HEK293 cells.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

Carbapenem-resistant Pseudomonas aeruginosa was designated as a critical priority pathogen by the World Health Organization (WHO) for which new therapeutic solutions are required. With the rapid dissemination of β-lactamases in P. aeruginosa, β-lactam (BL) antibiotics are used in conjunction with a β-lactamase inhibitor (BLI). The effectiveness of the BL/BLI combination could be further enhanced with the inclusion of an outer membrane (OM) permeabilizer, such as aminoglycosides and aminoglycoside-based adjuvants. Thus, the development of tobramycin derivatives reported herein focused on improving OM permeabilizing capabilities and reducing associated toxicity. The structure activity relationship (SAR) studies emphasized the effects of the nature of the cationic group; number of polar head groups and positive charges; and flexibility, length and steric bulk of the hydrophobic moiety. The optimized guanidinylated tobramycin-biphenyl derivative was non-cytotoxic and demonstrated the ability to potentiate ceftazidime and aztreonam monotherapy and in dual combinations with avibactam against multidrug-resistant and β-lactamase harboring isolates of P. aeruginosa. The triple combination of ceftazidime/avibactam plus guanidinylated tobramycin-biphenyl resulted in a rapid bactericidal activity within 4-8 hours of treatment, demonstrating the potential application of these guanidinylated amphiphilic tobramycin derivatives in augmenting BL/BLI.

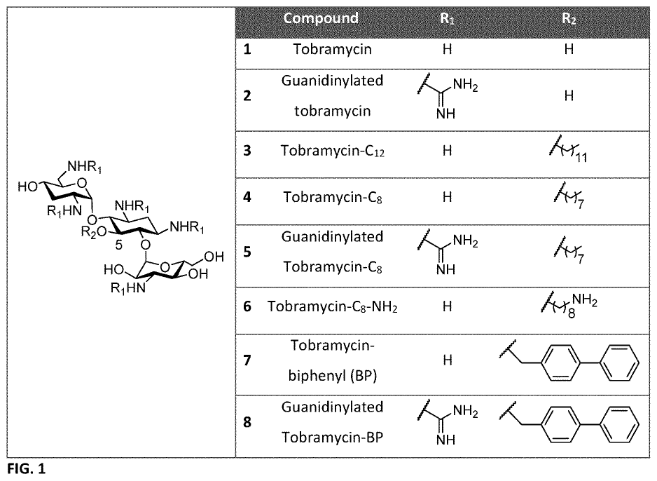

The structure activity relationship (SAR) studies of these conjugates have been reported, demonstrating the significance of the tether length, physiochemical properties of the secondary moiety, number of cationic groups and basicity (23). However, the attachment of a lipophilic group to render an amphiphilic tobramycin adjuvant, warrants further study. Thus, amphiphilic tobramycin derivatives were synthesized following established protocols (30, 31) and SAR (23), with the aim of improving OM permeabilizing properties and reducing inherent toxicity. The amenable C-5 position was chosen for the installment of several lipophilic moieties (FIG. 1). These include alkyl chains containing 8 and 12 carbon atoms which were shown to be optimal linker lengths (23) (FIG. 1). An 8-carbon long alkyl amine was included to mimic tobramycin derivatives with two positively charged head polar groups (21, 26) (FIG. 1). A biphenyl group was also chosen to discern the effects of a bulky, aromatic substituent (FIG. 1). The derivatives were further optimized by converting the amines to guanidinium groups (FIG. 1), which were shown to have enhanced antibiotic potentiation (24, 32). The guanidinium groups have a higher affinity for oxoanions such as phosphate groups due to its planar, Y-shaped geometry (33). In addition, the increased pKₐ imparted by the guanidinium groups allows it to remain protonated over a wider pH range which could theoretically facilitate electrostatic interactions with the lipid A phosphates (33). While not wishing to be bound to a particular theory or hypothesis, it is believed that a guanidinium group may show a higher potentiation such that there is a stronger interaction with the lipid A phosphates which may in turn result in stronger outer membrane destabilization effects.

The design and synthesis of tobramycin derivatives were directed by previous SAR, with a focus on enhancing OM permeabilization and reducing toxicity associated. Modifications were dedicated on altering the C-5 O-alkylated hydrophobic moiety and changing the nature of the cationic group. The optimized derivative 8, which conserved the ability to disrupt the OM and synergize with BL antibiotics, contained a biphenyl group and the amines converted to guanidinium groups. Compound 8 restored susceptibility of ceftazidime and aztreonam against MDR and β-lactamase harboring P. aeruginosa in both dual and triple combinations with avibactam. The triple combination of ceftazidime/avibactam plus 8 resulted in a bactericidal effect within 4-8 hours of treatment. An indication of the OM permeabilizing capability of 8 was substantiated by the time and adjuvant concentration-uptake of NPN, as well as the potentiation of rifampicin. In addition, 8 was non-cytotoxic against HEK293 cells up to approximately 106 times the active concentration.

According to another aspect of the invention, there is provided a method of treating a Pseudomonas aeruginosa infection comprising:
administering an effective amount of a compound having a formula as set forth below for compound 5 or compound 8 to an individual who has or is suspected of having a Pseudomonas aeruginosa infection

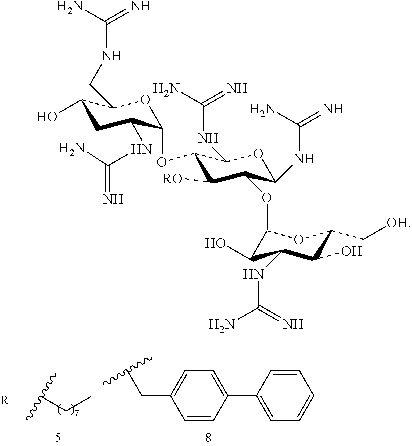

The Pseudomonas aeruginosa infection may be caused by a multi-drug resistant Pseudomonas aeruginosa strain.

The compound may be administered in combination with at least one antibiotic.

The antibiotic may be a β-lactam/β-lactamase antibiotic, for example, at least one β-lactam/β-lactamase antibiotic selected from the group consisting of ceftazidime, aztreonam, avibactam, piperacillin and meropenem.

In some embodiments, the compound is administered with ceftazidime and avibactam.

As will be appreciated by one of skill in the art and as discussed below, the compounds of the invention, for example, compounds 5 and 8, are administered in an effective amount, which is an amount that is sufficient to permeabilize the outer membrane of the Pseudomonas aeruginosa such that the effectiveness of any additional antibiotics administered is enhanced, as discussed herein.

Furthermore, when administered together with a suitable antibiotic, the compounds of the invention, for example, compounds 5 and 8, are administered together with the suitable antibiotic such that a bactericidal effect is observed.

The compounds of the invention are administered to the individual having or suspected of having the Pseudomonas aeruginosa infection until the desired bactericidal effect is observed and/or until the infection is sufficiently treated.

As used herein, “administering in combination” or “administering together” does not necessarily mean that the compounds of the invention are co-administered, that is, administered simultaneously, or co-formulated for simultaneous administration but that the compounds of the invention and the additional antibiotic(s) are administered such that their biological activities overlap.

According to another aspect of the invention, there is provided a method of permeabilizing a Gram Negative bacteria outer membrane comprising:
administering an effective amount of a compound having a formula as set forth below for compound 5 or compound 8 to a Gram Negative bacteria

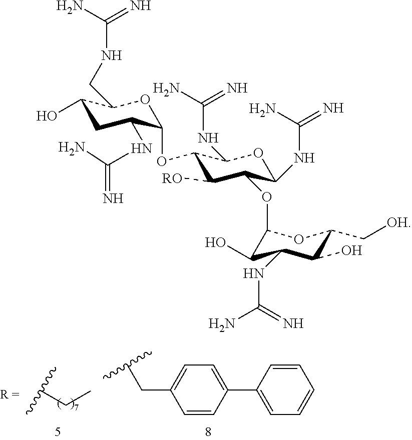

The Gram Negative bacteria may be Pseudomonas aeruginosa.

According to another aspect of the invention, there is provided a compound having a formula as set forth below for compound 5 or compound 8 for permeabilizing a Gram Negative bacteria outer membrane

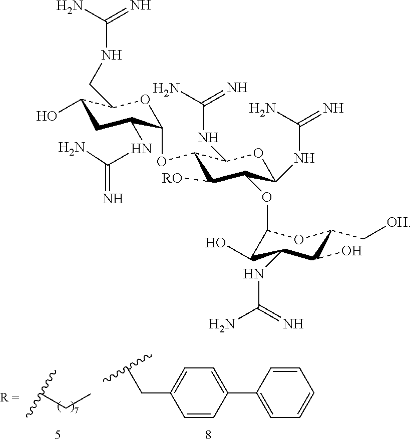

The invention will now be further explained and/or elucidated by way of examples; however, the invention is not necessarily limited to or by the examples.

### Example 1—Synthesis of Tobramycin Derivatives

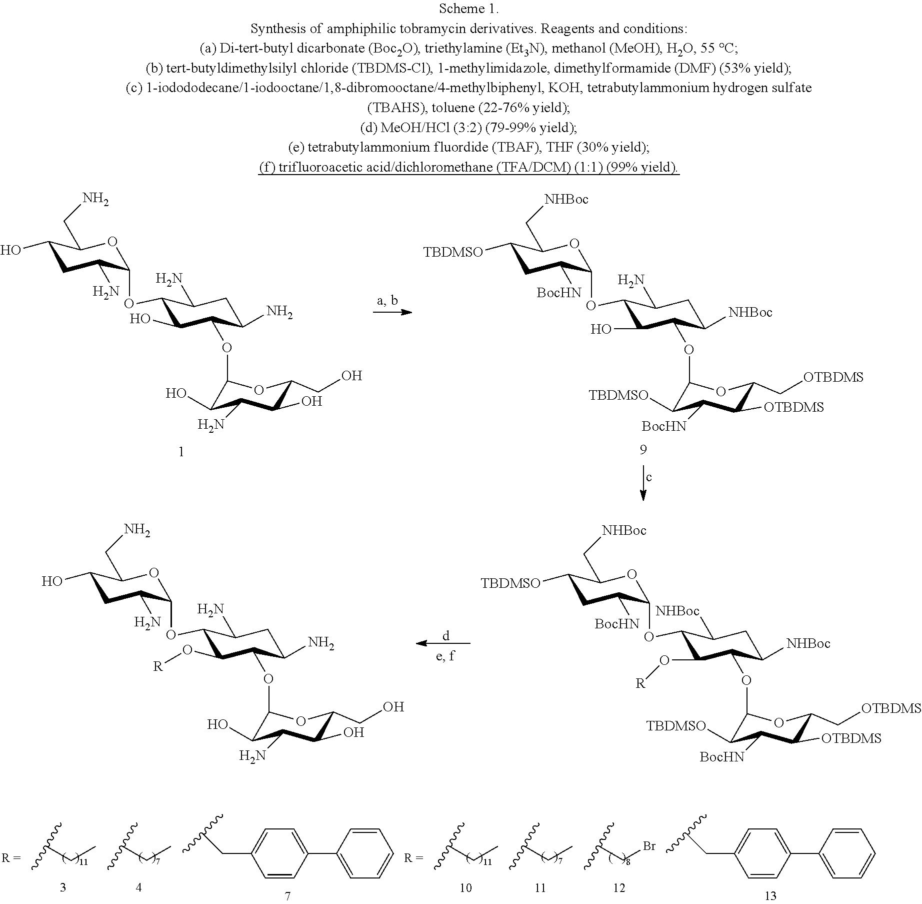

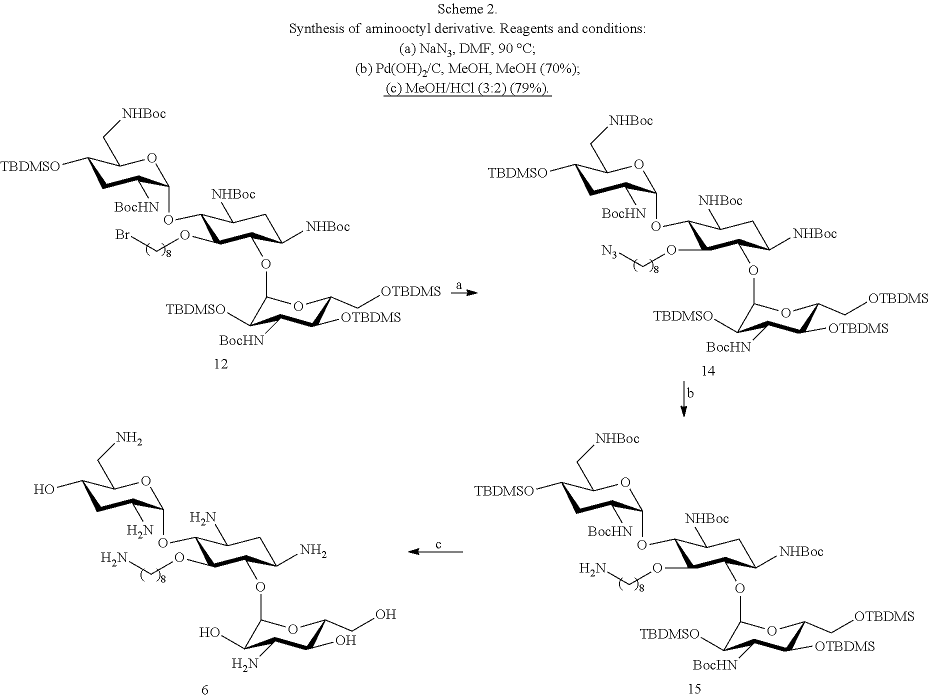

The preparation of the amphiphilic tobramycin derivatives was based on earlier established methods (30, 31, 34). This involved a protection strategy which was developed to conveniently leave the C-5 hydroxyl unreacted, wherein the amines were first protected with tert-butoxycarbonyl (Boc), and the alcohols were then protected with TBDMS (31) (Scheme 1). The resulting Boc and TBDMS-protected tobramycin 9 (31) was then used as an intermediate for the synthesis of the amphiphilic derivatives. Afterwards, a phase-transfer catalysis reaction was employed using the corresponding alkyl halide to alkylate the C-5 hydroxyl position with dodecyl (31), octyl, methyl biphenyl and octylbromide (34) (Scheme 1). The fully protected aminooctyl derivative 15 was synthesized as previously described (34), wherein the alkyl bromide was first converted to an azide using sodium azide, and the azide was reduced to an amine by hydrogenation with Pd(OH)₂/C (34) (Scheme 2). For the removal of the protecting groups, a single-step deprotection of the Boc and TBDMS groups using HCl/MeOH yielded 3 (31), 4 (Scheme 1) and 6 (Scheme 2). However, this reaction was not feasible for the biphenyl derivative. The electron density of the biphenyl presumably stabilizes the positive charge on the protonated ether oxygen, facilitating cleavage of the C—O bond. Thus, a stepwise selective deprotection of silyl ethers using TBAF/THF followed by the removal of Boc using TFA/DCM was utilized (35) to afford 7 (Scheme 1).

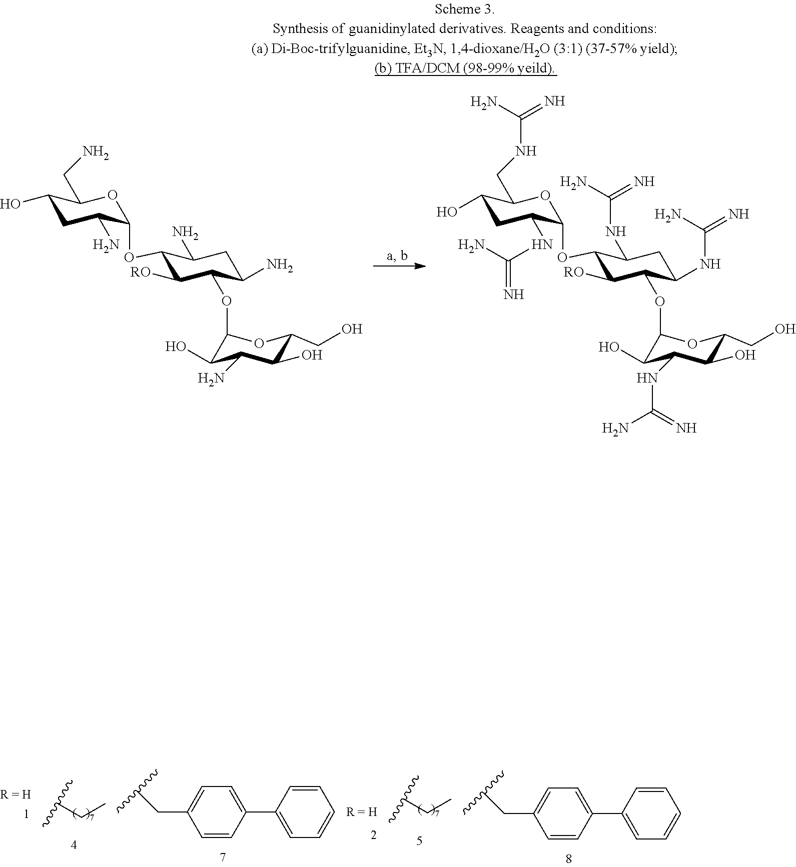

Lastly, the guanidinylated derivatives were prepared following a previously reported method (36). The efficient guanidinylation of 1 (36), 4 and 7 was carried out using Boc-triflyl guanidine (Scheme 3). The Boc groups were then removed using TFA/DCM to reproduce guanidinylated tobramycin 2 (36) and to generate the newly synthesized 5 and 8 (Scheme 3).

### Example 2—Antibacterial Activity of Tobramycin Derivatives

Following the completion of the synthesis, the broth microdilution assay was performed to assess the antibacterial activity of the derivatives against P. aeruginosa PAO1. The assay followed established protocols and the Clinical and Laboratory Standards Institute (CLSI) guidelines (37). The measured minimum inhibitory concentration (MIC) corresponds to the lowest concentration of the agent necessary to inhibit visible bacterial growth. In comparison to tobramycin, all derivatives exhibited an MIC of ≥128 μg/mL, indicating the loss of antibacterial activity. The reduced activity upon guanidinylation and/or alkylation of the C-5 hydroxyl group was consistent with previously reported findings. Selective conversion of either the 6′ or 6″ amines to guanidinium groups resulted in higher MICs than the parent tobramycin compound against PAO1 (38). Moreover, the absence of ribosomal binding activity via alkylation of the C-5 hydroxyl group was demonstrated by various tobramycin hybrids and conjugates (21, 23, 25, 26). These results indicate that guanidinylation and or alkylation of C-5 to tobramycin abolishes RNA-binding thereby changing the mode of action of tobramycin from a ribosomal target to the lipid A target of the outer membrane.

After establishing the MIC of the tobramycin derivatives, the ability of these adjuvants to synergize with BL antibiotics was investigated against P. aeruginosa PAO1. The checkerboard assays were conducted as previously described and in accordance with the CLSI guidelines (37). Synergy was defined by at least a 4-fold reduction in the MICs of both adjuvant and antibiotic. The panel of antibiotics are comprised of commonly used antipseudomonal agents and represent the different BL subclasses—cephalosporin (ceftazidime), monobactam (aztreonam), penicillin (piperacillin) and carbapenems (imipenem and meropenem) (12).

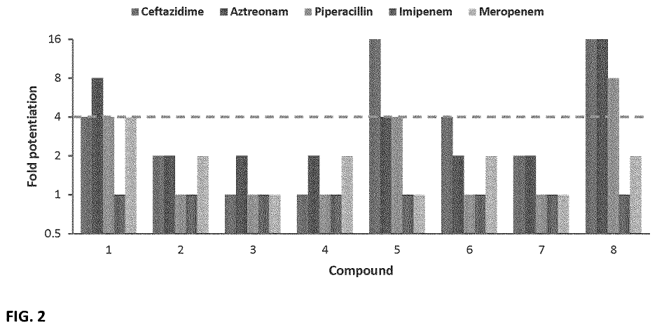

Among the amphiphilic derivatives, 6 potentiated ceftazidime by 4-fold (FIG. 2). Meanwhile, only the guanidinylated amphiphilic derivatives 5 and 8 potentiated ceftazidime, aztreonam and piperacillin by 4- to 16-fold (FIG. 2). In comparison, the parent aminoglycoside 1 potentiated ceftazidime, aztreonam, piperacillin and meropenem by 4- to 8-fold (FIG. 2). This result indicates that compounds 5 and 8 possess superior BL potentiation when compared to the parent tobramycin.

Based on these results, the additional positive charge in the aminooctyl derivative 6 is likely responsible for the modest potentiation of ceftazidime. However, it is evident that the combined effects of both the guanidinium groups and a lipophilic group at the C-5 position are essential for the synergism with most of the BL antibiotics. The guanidinium groups augmented the necessary electrostatic interactions with the LPS, leading to OM disruption. On the other hand, the hydrophobic portion is postulated to insert into the OM, further promoting entry of antibiotics (7). Conversely, these two structural features are likely insufficient to potentiate imipenem and meropenem. The observed additivity of the guanidinylated amphiphilic tobramycin with the carbapenems highlight the limitation of these adjuvants to further enhance antibiotic accumulation in the periplasm. While not wishing to be bound to a particular theory or hypothesis, one possible ne explanation of this result is that ceftazidime and aztreonam antibiotics possess reduced membrane permeability when compared to carbapenems. As a result, the observed potentiation effect of compound 5 or 8 will be greater when compared to carbapenems like meropenem.

The observed synergistic interactions of tobramycin with ceftazidime (39), aztreonam (40), piperacillin (41) and meropenem (42) conformed with previous studies. However, the mode of action between tobramycin and its amphiphilic derivatives are presumably different. Mechanism-based modelling experiments revealed that tobramycin disrupts the OM and consequently increases accumulation of the partner BL at the target site (41). It is also in part caused by both BL and tobramycin eliciting their antibacterial action by inhibiting cell wall and protein synthesis, respectively (20, 43). Meanwhile, the guanidinylated amphiphilic derivatives 5 and 8 which are devoid of antibacterial activity, selectively permeabilize the OM.

### Example 3—Synergy of Tobramycin Derivatives Against Multidrug-Resistant (MDR) Strains of P. aeruginosa

The synergism of the guanidinylated amphiphilic tobramycin derivatives with ceftazidime, aztreonam and piperacillin against PAO1 prompted further investigation against MDR clinical isolates of P. aeruginosa. Prior to the checkerboard assays, the MICs of 5 and 8 were determined to be >128 μg/mL against all strains tested.

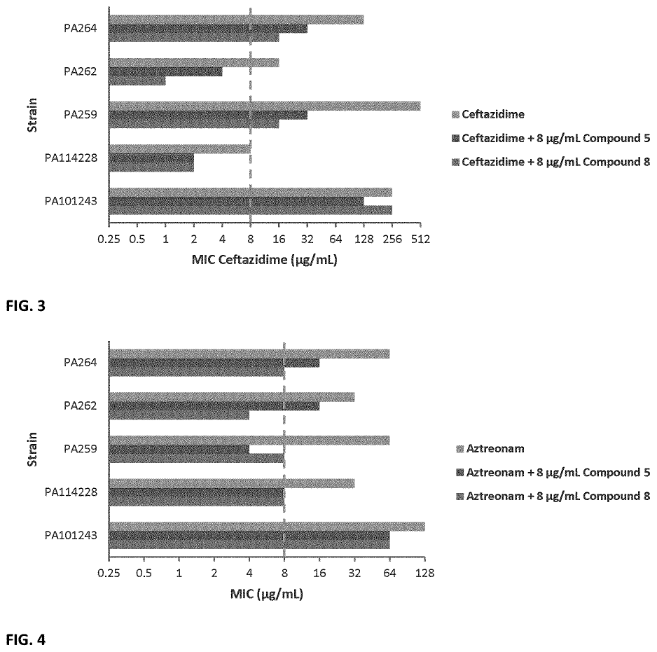

In combination with ceftazidime, 8 retained synergy with four strains, while 5 retained synergy with three strains (FIG. 3). Moreover, ceftazidime in combination with 5 or 8 attained the susceptibility breakpoint (≤8 μg/mL) against PA114228 and PA262 (FIG. 3).

Similarly, 8 maintained synergy with aztreonam against four strains, whereas 5 only maintained synergy with aztreonam against three strains (FIG. 4). In addition, aztreonam in combination with 8 reached the susceptibility breakpoint (≤8 μg/mL) against the four strains (FIG. 4). On the other hand, in combination with 5, the susceptibility breakpoint was only achieved against PA259 and PA114228 (FIG. 4). This result indicates that compounds 5 or 8 restore susceptibility of ceftazidime or aztreonam in most aztreonam- or ceftazidime-resistant isolates.

In contrast, synergy with piperacillin was only observed in a few strains. Piperacillin was potentiated by 8 against two strains and potentiated by 5 against PA262. The reduced potentiation of piperacillin in the clinical isolates is likely due to the presence of β-lactamases. The inactivation of most penicillin antibiotics by β-lactamases is the reason why piperacillin has limited use as a single agent and is exclusively combined with tazobactam (44, 45).

### Example 4—Synergy of Tobramycin Derivatives Against β-Lactamase Harboring Strains of P. aeruginosa

Since the main resistance mechanism against BL antibiotics is the hydrolysis by β-lactamase enzymes (46), it is imperative that the synergy evaluation is extended to P. aeruginosa strains containing these enzymes. Based on the results of the checkerboard assays against the MDR strains, the synergy of 5 and 8 with ceftazidime and aztreonam was further assessed against β-lactamase harboring P. aeruginosa isolates. These strains are characterized by their increased susceptibility to ceftazidime with the addition of avibactam. In addition, a triple combination therapy consisting of the tobramycin derivative, either BL antibiotic and avibactam was also investigated.

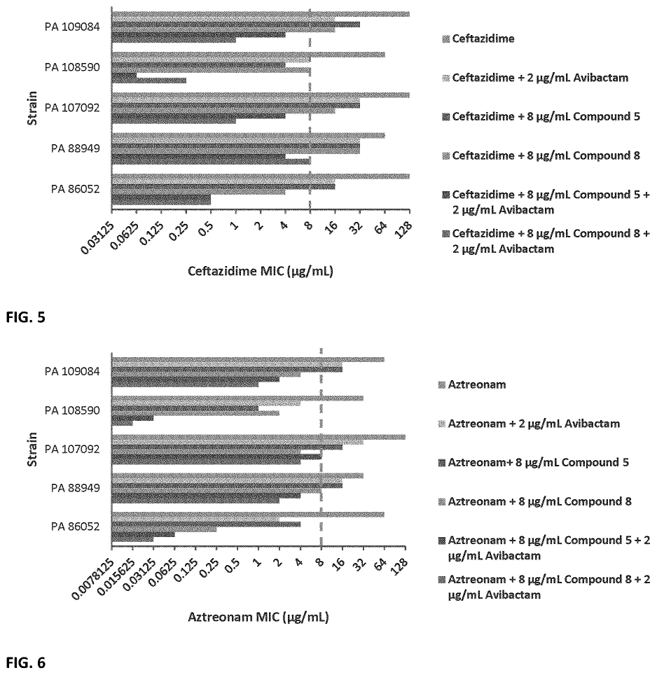

Compounds 5 or 8 were first verified to be inactive against the five strains, with MICs of 64 to >128 μg/mL. Ceftazidime monotherapy was also ineffective against all strains with MICs of 64-128 μg/mL (FIG. 5). Dual combination with either 5 and 8 potentiated ceftazidime by 4- to 32-fold, reducing its MIC to 4 to 32 μg/mL (FIG. 5). These results were comparable to the dual combination with avibactam, suggesting that the guanidinylated amphiphilic derivatives provide an additional benefit independent of avibactam in the presence of β-lactamase enzymes. This result indicates that compound 5 or 8 enhance the activity of ceftazidime with comparable potentiation as the clinically used ceftazidime/avibactam combination. It is then evident in the triple combination wherein either 5 and 8 plus avibactam, further reduced the MICs of ceftazidime to 0.063-4 μg/mL, which is below the susceptibility breakpoint (FIG. 5). In comparing the two derivatives, 5 and 8 demonstrated comparable potentiation of ceftazidime.

Similarly, aztreonam monotherapy was ineffective against the five strains with MICs of 32-128 μg/mL (FIG. 6). The dual combination of aztreonam with either 5 and 8 was either more effective or comparable to the dual combination with avibactam (FIG. 6). The triple combination restored the susceptibility of the isolates to aztreonam, with MICs of 0.016-8 μg/mL. 8 demonstrated a slightly higher fold-potentiation of aztreonam in most cases (FIG. 6). These results were similar to the potentiation of ceftazidime, wherein OM permeabilization of the guanidinylated amphiphilic derivatives and β-lactamase inhibition of avibactam, act synergistically to further enhance the potency of aztreonam. This result indicates that compound 5 or 8 enhance the activity of aztreonam with comparable potentiation as the ceftazidime/avibactam combination in Phase 3 clinical trials.

### Example 5—Time-Kill Kinetics Assay

To elucidate the mechanistic killing of the triple combination studies, the time-kill kinetics assay was performed as previously described (32). The study focused on the clinically approved ceftazidime/avibactam combination with the inclusion of 8, the derivative which demonstrated higher antibiotic potentiation. 8 μg/mL of ceftazidime and 2 μg/mL avibactam were used to reflect the clinically used 4:1 ratio. 8 μg/mL of 8 was designated as it was the lowest concentration wherein synergy was observed in the checkerboard assays.

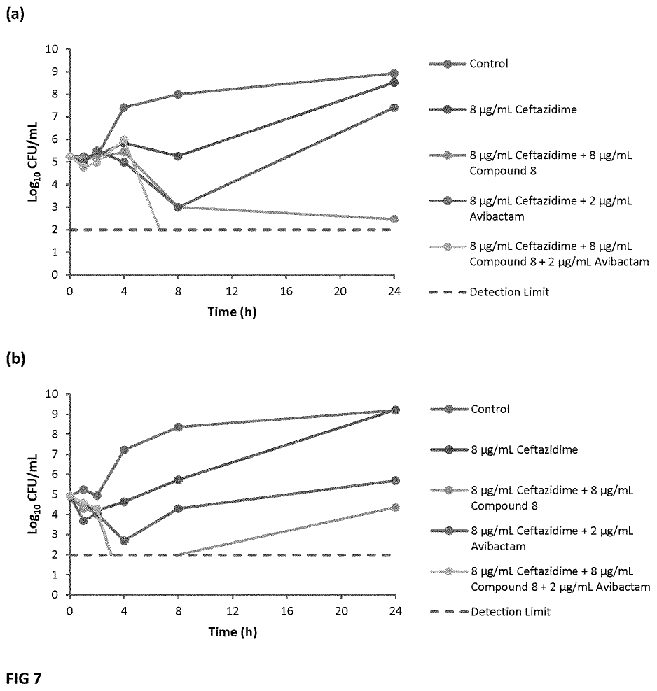

Against PA 86052, ceftazidime monotherapy and ceftazidime/avibactam did not exhibit any antibacterial effect, with growth similar to the control over the 24-hour time period (FIG. 7a). Meanwhile, ceftazidime with compound 8 demonstrated a bacteriostatic effect. Lastly, the triple combination of ceftazidime/avibactam plus 8 displayed a rapid bactericidal effect with >3 log reduction within 8 hours (FIG. 7a). Against PA 107092, ceftazidime monotherapy was ineffective and allowed bacterial growth similar to the control, while both dual combination therapies exhibited a bacteriostatic effect over a period of 24 hours (FIG. 7b). Only the triple combination resulted in bacterial killing which occurred at the 4-hour mark with a 3 log reduction (FIG. 7b). The dual combination with 8 initially resulted in a reduction of the bacterial load at 4 hours analogous to the triple combination, but regrowth occurred at 24 hours.

The synergy of 8 with ceftazidime against PA 86052 and PA 107092 determined in the checkerboard assays corresponded to growth inhibition based on the time-kill kinetics experiment. This combination being more effective than ceftazidime/avibactam in the checkerboard assays was also translated in the time-kill curve. Ceftazidime/avibactam resulted in higher bacterial loads of PA 86052 and demonstrated growth inhibitory effects against PA 107092, but at a rate of 1 log slower than the dual combination with 8. This result indicates that the triple combination comprising of 8/ceftazidime/avibactam is superior to the clinically used ceftazidime/avibactam combination. On the other hand, the encouraging results of ceftazidime/avibactam with 8 in the checkerboard assays equated to bacterial killing within 4 to 8 hours of treatment.

The outcome of the time-kill data suggests that the incorporation of both 8 and avibactam is necessary to elicit a bactericidal action. The OM permeabilizer enhances the accumulation of BL antibiotics in the periplasm, which could account for the inactivation of BL antibiotics due to the saturation of β-lactamase enzymes.

### Example 6—NPN Uptake Assay

The assumed mechanism by which these tobramycin derivatives potentiate antibiotics is through OM permeabilization, as detailed by other tobramycin-based adjuvants. To verify this, the uptake of 1-N-phenylnaphthylamine (NPN), a membrane-impermeable probe, was analyzed. Owing to its hydrophobic nature, NPN is initially excluded from the OM unless the integrity of the OM is disrupted (47). NPN then strongly fluoresces as it enters the phospholipid bilayer, and the measured change in fluorescence is indicative of OM perturbation (47). The analyses were tested on P. aeruginosa PAO1 cells with the gold standard OM permeabilizer PMBN used as a control. 6 μM was set as the max concentration which was equivalent to ~8 μg/mL, the active concentration of compounds 5 and 8.

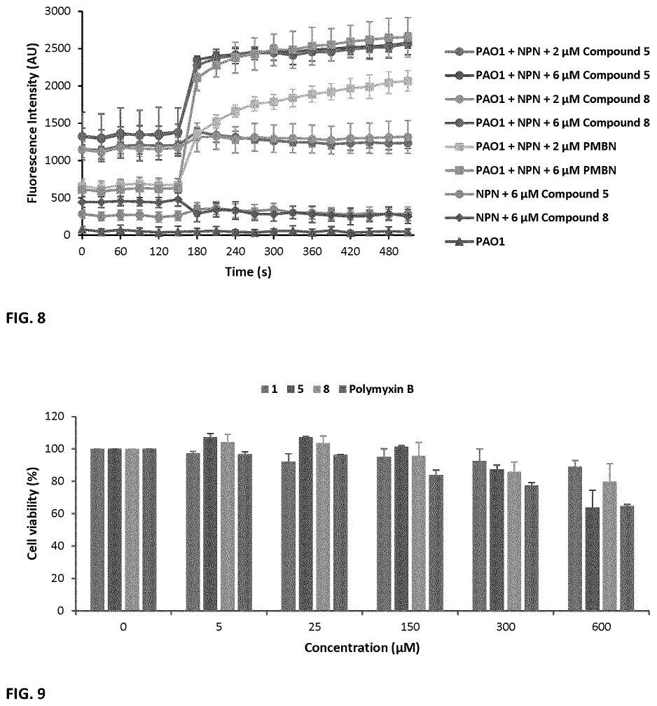

Cells treated with 6 μM of 5 and 8 resulted in a change in fluorescence intensity at 150 s upon addition of NPN, denoting the uptake of the dye in a time-dependent manner (FIG. 8). In addition, the minimal change in fluorescence observed at the lower concentration of 2 μM signifies the concentration-dependent uptake of NPN (FIG. 8). These results resembled the fluorescence difference exhibited by PMBN, suggesting increased permeation of NPN as a consequence of OM disruption. Overall, the NPN study indicates that compounds 5 or 8 destabilize the outer membrane of Pseudomonas in a concentration-dependent manner like the gold standard outer membrane permeabilizer PMBN.

### Example 7—Potentiation of Rifampicin

Another evidence of OM permeabilization is the ability of the guanidinylated amphiphilic tobramycin derivatives to synergize with membrane-impermeable antibiotics such as rifampicin. 5 and 8 potentiated rifampicin by 16- and 64-fold, respectively, against PAO1. Rifampicin has limited capacity in crossing the OM of Gram-negative bacteria, and is restricted to treating infections caused by Mycobacteria and Gram-positive cocci (48). Unlike the BL antibiotics that permeate the OM through a porin-mediated uptake, the large molecular weight and hydrophobicity of rifampicin make it unable to enter the narrow porins, and rely on slow passive diffusion to traverse the lipid bilayer (49). Therefore, the augmented potency of rifampicin against P. aeruginosa is linked to its increased intracellular concentration through OM destabilization.

### Example 8—Cytotoxicity Assay

Aminoglycoside use is associated with ototoxicity (50) and nephrotoxicity (51). Therefore, the development of tobramycin-based derivatives should also be aimed to mitigate these side effects. Aminoglycoside-induced nephrotoxicity is facilitated by the cationic nature of these compounds (52-54). However, the challenge with cationic amphiphiles is that the introduction of the lipophilic group is also a source of toxicity through nonspecific binding and hemolytic activity (55). Therefore, determining which hydrophobic moiety to install is necessary to minimize the toxicity.

As an initial screening of the toxicity, the cell viability of the tobramycin derivatives was assessed in human embryonic kidney (HEK) 293 cell lines. Polymyxin B was utilized as negative control and by being a membrane permeabilizing amphiphile resembling the tobramycin derivatives. Doxorubicin, an anticancer agent, served as positive control, which displayed 0% cell viability at a concentration of 125 μM.

Among the amphiphilic derivatives, 3 was highly toxic relative to tobramycin, causing cell death at 600 μM. In contrast, the less hydrophobic 4 and 7 were comparable to 1 and maintained 86.05-100.88% cell viability up to 600 μM. The observed trend was in agreement with earlier studies involving amphiphilic neamine derivatives, wherein an increase in lipophilicity correlated to an increase in cytotoxicity (29). Regarding the aminooctyl derivative, the cell viability at 600 μM of 6 was lower in comparison to 4. This suggests that the additional positive charge introduces some toxicity, which coincides with data on truncated polymyxin derivatives with fewer charges (e.g., polymyxin B nonapeptide (PMBN) and SPR741 (56)). In comparing the guanidinylated derivatives to their non-guanidinylated counterparts, a slight decrease in the cell viability at higher concentrations was observed for 2 and 8. Conversely, in the case of 5, the cell viability greatly decreased to 63.75% at 600 μM (FIG. 9). Recently published data on guanidinylated polymyxins also indicated that guanidinylation contributed to cytotoxicity (24). However, it is worth noting that the guanidinylated amphiphilic tobramycin derivatives possess lower cytotoxic effects when compared to the guanidinylated polymyxins against the same cell line. Overall, these results indicate that compound 8 possesses reduced cytotoxicity than PMB and compound 5 possesses comparable toxicity at 600 μM concentration against HEK 293 cell lines. The reduced cytotoxicity of 8 when compared to PMB is highly desired. Lead compound, 8, maintained 79.84% cell viability at 600 μM (FIG. 8) which corresponds to 848.04 μg/mL. This is approximately 106 times the active concentration of 8 μg/mL used in the biological assays. In comparison, PMB maintained 64.7% cell viability.

### Example 9—Experimental Section

### Chemistry

All reagents and chemicals were purchased from Sigma-Aldrich (United States) except N,N′-bis(tert-butoxycarbonyl)-N″-trifylguanidine purchased from Biosynth (United States), and 4-bromomethylbiphenyl purchased from AK Scientific (United States). The reagents were used without further purification. Reactions were monitored using thin-layer chromatography (TLC) on Merck (Germany) 0.25 mm Silica gel 60 F254 plates and visualized under UV light and/or by staining with ninhydrin in butanol and H₂SO₄ in MeOH solutions. Compounds were purified by normal and reverse-phase flash chromatography using Silicycle (Canada) Silica Flash G60 (60-200 μm) and C18 (17% C) (40-63 μM), respectively, and/or an automated system using Biotage (Sweden) Selekt Flash instrument with Biotage Sfär columns. ¹H, ¹³C and 2D NMR experiments were recorded on Bruker (Germany) AMX-300, AMX-400 and AMX-500 spectrometers. MALDI-MS analyses were performed in positive ion mode with 2,5-dihydrobenzoic acid as matrix on Bruker Ultraflextreme MALDI—time of flight (TOF)/TOF mass spectrometer.

### General Procedure A: C-5 O-Alkylation of Boc and TBDMS Protected Tobramycin

Compound 9 (1 mmol) was dissolved in toluene. KOH (3 mmol), alkylhalide (3 mmol) and a catalytic amount of tetrabutylammonium hydrogen sulfate (TBAHS) (0.1 mmol) was added to the solution. The reaction was stirred at room temperature for 16-18 hours. Water was added to the reaction mixture and extracted thrice with ethyl acetate. The combined organic extracts were washed with saturated brine and dried over anhydrous Na₂SO₄. The crude mixture was then purified using flash chromatography with an elution gradient of 100% hexanes to 10:1 (v/v) hexanes:ethyl acetate.

### Synthesis and Characterization of 5-O-(Octyl)-1,3,2′,6′,3″-penta-N-Boc-4′,2″,4″,6″-tetra-O-TBDMS-tobramycin

Compound 11 was synthesized following general procedure A with 1-iodooctane as the alkyl halide to obtain desired product as white solid (12% yield). ¹H NMR (500 MHz, CDCl₃) 5.21 (m, 1H, 1′), 5.14 (m, 1H, 1″), 5.06-5.04 (m, 1H), 4.77 (s, 1H), 4.50 (s, 1H), 4.26-4.06 (m, 3H), 3.78-3.16 (m, 14H), 2.45 (m, 1H), 2.02-1.93 (m, 1H), 1.53-1.38 (m, 45H, Boc t-Bu), 1.30-1.23 (m, 12H, b-g), 0.94-0.85 (m, 39H, h, TBDMS t-Bu), 0.14-0.02 (m, 24H, TBDMS Si-Me). ¹³C NMR (126 MHz, CDCl₃) δ 155.50, 154.72, 154.54, 96.54, 85.69, 79.88, 79.35, 79.19, 75.26, 73.39, 72.66, 67.97, 66.80, 63.08, 57.28, 50.51, 48.96, 48.37, 41.64, 36.63, 35.62, 31.86, 30.59, 29.94, 29.66, 29.28, 28.62, 28.49, 28.48, 28.38, 26.14, 26.12, 26.00, 25.97, 25.77, 24.67, 23.40, 22.63, 18.47, 18.32, 18.08, 17.90, 14.07, −3.44, −3.82, −4.22, −4.89, −4.96, −5.10, −5.21, −5.26.

### Synthesis and Characterization of 5-O-(4-Methylbiphenyl)-1,3,2′,6′,3″-penta-N-Boc-4′,2″,4″,6″-tetra-O-TBDMS-tobramycin

Compound 13 was synthesized following general procedure A with 1-bromomethylbiphenyl as the alkyl halide to obtain desired product as a white solid (41% yield). ¹H NMR (400 MHz, CDCl₃) δ 7.60-7.58 (m, 2H, biphenyl), 7.53-7.51 (m, 2H, biphenyl), 7.47-7.43 (m, 4H, biphenyl), 7.37-7.33 (m, 1H, biphenyl), 5.39 (m, 1H), 5.27-5.25 (m, 2H), 5.13-5.11 (m, 1H), 4.86 (m, 1H), 4.62-4.59 (m, 1H), 4.30-4.26 (m, 2H), 3.90-3.87 (m, 2H), 3.74-3.36 (m, 10H), 3.20 (m, 1H), 2.56-2.53 (m, 1H), 2.07-2.04 (m, 1H), 1.49-1.28 (m, 45H, Boc t-Bu), 0.97-0.84 (m, 36H, TBDMS t-Bu), 0.18-0.07 (m, 18H, TBDMS Si-Me), −0.02-−0.08 (m, 6H, TBDMS Si-Me). ¹³C NMR (126 MHz, CDCl3) δ 155.64, 141.16, 129.10, 128.66, 127.06, 127.00, 79.94, 79.43, 75.77, 67.15, 63.67, 28.64, 28.54, 28.51, 28.27, 26.14, 26.05, 25.96, 25.79, 18.43, 18.33, 18.12, 17.93, 1.00, −3.60, −4.23, −4.90, −5.26.

### General Procedure B: Global Deprotection of Amine and Hydroxyl Groups

The Boc and TBDMS-protected C-5 alkylated tobramycin compounds were treated with 40% HCl in MeOH at room temperature for 1-3 hours. The methanolic HCl solution was removed at reduced pressure. 2% MeOH in ether was then added to the resulting residue, stirred for 1 minute and the solvent decanted. The crude product was purified using reverse-phase flash chromatography eluted with 100% deionized water.

### Characterization of 5-O-(Octyl)-tobramycin

Compound 4 was synthesized following general procedure B to obtain desired product as a white solid (98% yield). 1H NMR (500 MHz, D2O): δ 5.40 (d, 1H, 1′), 5.19 (d, 1H, 1″), 4.30 (m, 1H, 5′), 4.21 (dd, 1H, 4), 3.98-3.89 (m, 5H, 6, 4′, 2″, 6″, a), 3.87-3.79 (m, 4H, 5, 4″, 5″, 6″), 3.77-3.72 (m, 2H, 2′, a), 3.67-3.57 (m, 3H, 1, 3, 3″), 3.45-3.40 (m, 1H, 6′), 3.35-3.32 (dd, 1H, 6′), 2.58-2.54 (m, 1H, 2), 2.31-2.23 (m, 2H, 3′), 2.05-1.98 (m, 1H, 2), 1.66 (br s, 2H, b), 1.29 (m, 10H, c-g), 0.88 (t, 3H, h). 13C NMR (125 MHz, D2O): δ 101.37 (1″), 92.72 (1′), 81.86, 81.82, 76.70, 75.80, 73.83 (a), 73.16, 68.55, 64.79, 63.21, 59.23, 54.78, 49.79, 48.46, 47.31, 38.53, 31.13, 29.45 (b), 28.90, 28.53, 28.10, 27.75, 25.30, 22.08, 13.49 (h). MALDI-MS m/z calculated: 602.27. measured m/z 602.395 [M+Na]+.

### Characterization of 5-O-(8-Aminooctyl)-tobramycin

Compound 6 was synthesized following general procedure B to obtain desired product as a white solid (79% yield). ¹H NMR (500 MHz, D₂O) δ 5.41-5.40 (d, 1H, 1′), 5.21-5.20 (d, 1H, 1″), 4.32-4.29 (m, 1H, 5′), 4.26-4.24 (m, 1H, 4), 3.99-3.86 (m, 9H), 3.84-3.79 (m, 2H), 3.77-3.73 (m, 3H, 2′ a), 3.69-3.58 (m, 3H, 1, 3, 3″), 3.47-3.42 (m, 1H, 6′), 3.36-3.32 (m, 1H, 6′), 3.03-3.00 (m, 2H), 2.72-2.54 (m, 1H, 2), 2.33-2.23 (m, 2H, 3′), 2.07-2.00 (m, 1H, 2), 1.71-1.61 (m, 4H, b, g), 1.41-1.30 (m, 8H, c-f). ¹³C NMR (126 MHz, D₂O) δ 103.42, 101.35, 92.74, 92.07, 82.69, 81.87, 81.82, 76.75, 76.66, 75.75, 74.98, 73.77, 73.53, 73.15, 72.65, 68.58, 68.18, 66.07, 64.82, 63.87, 63.26, 59.27, 57.95, 57.53, 54.81, 49.90, 49.80, 49.23, 48.98, 48.49, 47.56, 47.35, 39.62, 39.34, 38.58, 32.73, 29.49, 28.83, 28.51, 28.33, 28.25, 28.12, 27.91, 27.73, 26.79, 25.68, 25.64, 25.42, 25.22, 25.18. MALDI-MS m/z calculated: 617.39. measured m/z 617.427 [M+Na]+.

### General Procedure C: Guanidinylation of C5 O-Alkylated Tobramycin

The C-5 O-alkylated tobramycin was dissolved in water (0.5 mL). N,N′-Bis(tert-butoxycarbonyl)-N″-trifylguanidine (15 eq.) and 1,4-dioxane (2.5 mL) were then added in alternating portions, ensuring that the solution remained relatively clear. After 5 min, Et₃N (15 eq.) was added, and the reaction was stirred for 3-4 days at RT. 1,4-dioxane was removed under reduced pressure. The remaining residue was extracted thrice with DCM. The organic layers were combined, dried over anhydrous Na₂SO₄ and concentrated under reduced pressure.

### Synthesis and Characterization of 5-O-(Octyl)-guanidinoBoc₁₀-tobramycin

Boc protected guanidinylated Tob-C₈ was synthesized following general procedure C. The crude product was purified using an automated flash chromatography system with an elution gradient of 0-10% MeOH in DCM to obtain desired product as a white solid (57% yield). ¹H NMR (500 MHz, CDCl₃) δ 11.61 (s, 1H, Boc guanidine), 11.50-11.34 (m, 4H, Boc guanidine), 8.90-8.89 (d, 1H, guanidine), 8.55-8.53 (m, 1H, guanidine), 8.50-8.47 (m, 1H, guanidine), 8.45-8.43 (1H, m, guanidine), 8.23-8.21 (m, 1H, guanidine), 5.41-5.40 (d, 1H), 5.20-5.19 (d, 1H), 5.05 (m, 1H), 4.51-4.42 (m, 3H), 4.06-4.02 (m, 1H), 3.95-3.86 (m, 3H), 3.82-3.70 (m, 4H), 3.60-3.53 (m, 3H), 3.50-3.44 (m, 2H), 3.37-3.35 (m, 1H), 3.27-3.24 (m, 1H), 3.04-3.02 (m, 1H), 2.34-2.30 (m, 1H), 2.16-2.12 (m, 2H), 1.68-1.58 (m, 2H, b), 1.54-1.51 (m, 18H, Boc t-Bu), 1.50-1.47 (m, 54H, Boc t-Bu), 1.45-1.42 (18H, Boc t-Bu), 1.27-1.19 (m, 10H, c-g), 0.84-0.83 (t, 3H, h). ¹³C NMR (126 MHz, CDCl₃) δ 163.72, 163.43, 163.35, 162.72, 162.10, 158.30, 157.21, 155.95, 155.68, 155.52, 153.20, 153.17, 152.83, 152.81, 152.68, 148.63, 97.05, 95.49, 83.62, 83.58, 83.38, 83.29, 83.13, 79.61, 79.53, 79.26, 79.20, 79.14, 79.12, 75.98, 73.69, 73.05, 72.48, 71.80, 69.79, 63.68, 62.73, 57.82, 49.45, 48.46, 48.27, 41.33, 31.83, 31.62, 31.21, 29.74, 29.56, 29.15, 28.27, 28.21, 28.17, 28.14, 28.03, 28.00, 27.96, 27.85, 25.54, 22.62, 14.09, 1.00.

### Synthesis and Characterization of 5-O-(4-Methylbiphenyl)-guanidinoBoc₁₀-tobramycin

Boc protected guanidinylated Tob-BP was synthesized following general procedure C. The crude product was purified using an automated flash chromatography system with an elution gradient of 0-10% MeOH in DCM to obtain desired product as a white solid (37% yield). ¹H NMR (500 MHz, CDCl₃) δ 11.49-11.34 (m, 5H, Boc guanidine), 8.82-8.81 (m, 1H, guanidine), 8.61-8.48 (m, 3H, guanidine), 8.30-8.29 (m, 1H, guanidine), 7.55-7.51 (m, 4H), 7.51-7.34 (m, 4H), 7.30-7.27 (m, 1H), 5.48 (d, 1H), 5.24-5.22 (d, 1H), 5.12 (m, 1H), 5.03-5.01 (m, 1H), 4.92-4.84 (m, 2H), 4.61-4.54 (m, 1H), 4.42-4.32 (m, 1H), 4.08-3.92 (m, 4H), 3.89-3.85 (m, 2H), 3.79-3.67 (m, 3H), 3.63-3.57 (m, 1H), 3.53-3.43 (m, 2H), 3.40-3.24 (m, 4H), 3.16-3.14 (m, 1H), 2.39-2.34 (m, 1H), 2.17-2.12 (m, 1H), 1.55-1.37 (m, 90H, Boc t-Bu). ¹³C NMR (126 MHz, CDCl₃) δ 163.33, 162.70, 162.27, 158.57, 155.98, 155.36, 153.20, 152.95, 152.82, 141.00, 140.26, 128.54, 127.35, 127.15, 127.10, 97.61, 83.64, 83.55, 83.38, 83.17, 79.65, 79.59, 79.35, 79.24, 79.06, 78.38, 77.25, 77.20, 76.99, 76.74, 73.22, 72.94, 71.52, 69.45, 66.41, 63.64, 62.01, 48.56, 48.28, 28.30, 28.27, 28.25, 28.20, 28.18, 28.16, 28.13, 28.07, 28.01, 27.97.

### Synthesis and Characterization of 5-O-(4-Methylbiphenyl)-1,3,2′,6′,3″-penta-N-Boc-tobramycin

Compound 13 was dissolved in tetrahydrofuran (THF) (5 mL) and tetrabutylammonium fluoride (TBAF) (6 eq.) was added and stirred under N₂ for 2 hours. The reaction mixture was concentrated in vacuo, dissolved in water and extracted thrice with dichloromethane (DCM). The organic layers were combined, dried over anhydrous Na₂SO₄ and concentrated in vacuo. The crude product was purified using flash chromatography with 40:1 DCM:MeOH (v/v) to obtain desired product as a white solid (30% yield). ¹H NMR (500 MHz, MeOD) δ 7.57-7.55 (m, 2H, biphenyl), 7.53-7.51 (m, 2H, biphenyl), 7.48-7.44 m, 2H, biphenyl), 7.41-7.38 (m, 2H, biphenyl), 7.31-7.28 (m, 1H, biphenyl), 5.36-5.31 (m, 2H), 5.12-5.10 (d, 1H), 4.87-4.85 (d, 1H), 3.79-3.33 (m, 15H), 2.09-2.07 (m, 1H), 1.88-1.85 (m, 1H), 1.84-1.57 (m, 1H), 1.47-1.39 (m, 36H, Boc t-Bu), 1.30-1.18 (m, 9H, Boc t-Bu). ¹³C NMR (126 MHz, MeOD) δ 157.98, 156.61, 156.07, 140.88, 140.08, 128.36, 127.79, 126.80, 126.47, 126.42, 98.46, 95.85, 84.74, 79.51, 78.99, 77.10, 73.63, 73.02, 71.98, 70.61, 65.73, 60.69, 55.90, 48.98, 48.74, 40.82, 33.52, 27.47, 27.42, 27.39, 27.29.

### General Procedure D: Global Deprotection of Amine Groups

The Boc-protected tobramycin derivative was dissolved in DCM (2 mL) and treated with trifluoroacetic acid (TFA) (2 mL). The reaction was stirred at room temperature for 1-2 hours and concentrated under reduced pressure. 2% MeOH in diethyl ether was then added to the resulting residue, stirred for 1 minute and the solvent was decanted. The crude product was purified using reverse-phase flash chromatography eluted with 100% deionized water.

### Characterization of 5-O-(4-Methylbiphenyl)-tobramycin

Compound 7 was synthesized following general procedure D to obtain desired product as a white solid (99% yield). ¹H NMR (500 MHz, D₂O) δ 7.66-7.60 (dd, 4H, c-f), 7.46-7.41 (m, 5H, i-l), 7.35-7.32 (m, 1H, m), 5.18-5.17 (d, 1H, 1′), 5.10-5.08 (m, 1H, a), 5.01 (m, 1H, 1″), 4.12-4.04 (m, 2H, 4, 5′), 3.89-3.82 (m, 3H), 3.77-3.75 (m, 1H), 3.70-3.63 (m, 2H), 3.58-3.53 (m, 2H), 3.47-3.43 (m, 2H), 3.37-3.34 (m, 1H), 3.11-3.06 (m, 1H), 3.02-2.99 (m, 1H), 2.91-2.90 (m, 1H), 2.44-2.42 (m, 1H), 1.91-1.71 (m, 1H, 2, 3′). ¹³C NMR (126 MHz, D₂O) δ 163.18, 162.90, 140.78, 139.85, 136.84, 129.34, 128.15, 127.41, 126.95, 117.59, 115.27, 101.65, 92.86, 82.61, 82.55, 77.34, 76.63, 74.95, 73.34, 68.55, 64.84, 62.76, 59.10, 54.80, 49.81, 48.39, 46.79, 38.03, 27.70. MALDI-MS m/z calculated: 634.34. measured m/z 634.345 [M+H]⁺.

### Characterization of 5-O-(Octyl)-guanidinotobramycin

Compound 5 was synthesized following general procedure D to obtain desired product as a white solid (99% yield). ¹H NMR (500 MHz, D₂O) δ 5.34 (d, 1H, 1′) 5.13-5.09 (d, 1H, 1″), 3.79-3.57 (m, 8H), 3.55-3.39 (m, 7H), 3.37-3.35 (m, 1H), 3.33-3.29 (m, 1H), 2.12-2.05 (m, 2H), 1.60-1.50 (m, 2H), 1.43-1.34 (m, 2H, b), 1.13-1.05 (m, 10H, c-g), 0.71-0.68 (m, 3H, h). ¹³C NMR (126 MHz, D2O) δ 163.46, 163.18, 162.89, 162.61, 158.59, 157.99, 156.65, 156.40, 155.94, 119.91, 117.59, 115.27, 112.94, 98.02, 94.79, 83.63, 78.20, 76.66, 74.68, 72.27, 72.06, 69.67, 67.70, 63.71, 60.07, 57.52, 51.89, 50.47, 49.07, 41.77, 32.85, 32.43, 30.95, 29.30, 28.69, 28.30, 24.89, 22.02, 13.42. MALDI-MS m/z calculated: 790.49. measured m/z 790.502 [M+H]⁺.

### Characterization of 5-O-(4-Methylbiphenyl)-guanidinotobramycin

Compound 8 was synthesized following general procedure D to obtain desired product as a white solid (99% yield). ¹H NMR (500 MHz, D₂O) δ 7.57-7.52 (m, 4H, c-f), 7.40-7.37 (m, 2H), 7.31-7.26 (m, 3H), 5.39 (d, 1H, 1′) 5.14-5.13 (d, 1H, 1″), 5.04-5.01 (d, 1H), 4.70-4.67 (d, 1H), 3.87-3.80 (m, 2H), 3.71-3.63 (m, 3H), 3.60-3.54 (m, 1H), 3.52-3.42 (m, 3H), 3.40-3.25 (m, 6H), 2.18-2.14 (m, 1H), 1.97-1.93 (m, 1H), 1.64-1.49 (m, 2H). ¹³C NMR (126 MHz, D₂O) δ 163.20, 162.92, 158.51, 157.94, 156.73, 156.52, 155.46, 140.27, 139.68, 129.28, 127.88, 126.92, 126.88, 126.10, 117.58, 115.26, 98.20, 95.55, 83.08, 78.37, 77.42, 73.99, 72.08, 71.92, 69.46, 67.62, 63.89, 59.70, 57.46, 52.03, 50.55, 48.92, 32.35. MALDI-MS m/z calculated: 844.45. measured m/z 844.43 [M+H]+. MALDI-MS m/z calculated: 866.44. measured m/z 866.405 [M+Na]⁺

### Bacterial Isolates and Growth Conditions

All antibiotics were purchased from commercial sources. Bacterial isolates were obtained from the American Type Culture Collection (ATCC), and from the Canadian National Intensive Care Unit (CAN-ICU) (59) and Canadian Ward (CANWARD) surveillance studies (60). CAN-ICU and CANWARD clinical isolates were collected from patients with presumed infectious diseases admitted in participating Canadian medical centers either to the intensive care unit or medical wards. Prior to microbiological testing, bacterial cultures were grown overnight in lysogeny broth (LB) at 37° C., shaking at 250 rpm.

### Antimicrobial Susceptibility Assay

The broth microdilution assay was performed in accordance with standard protocols as previously described (61). An inoculum of the culture was diluted in 0.85% saline solution to attain 0.5 McFarland turbidity. The inoculum was further diluted in Mueller-Hinton broth (MHB) for inoculation to a final concentration of 5×10⁵ colony forming units/mL (CFU/mL). The agents were serially diluted two-fold in CA-MHB in a 96-well plate. Equal volumes of bacterial solution were added to the designated wells. The well containing only broth served as negative control, while the well containing the broth and bacterial solution served as positive control. The plate was then incubated at 37° C. for 18 hours. After incubation, Molecular Devices (United States) Emax Plus microplate reader was used to measure the optical density (OD) at a wavelength of 590 nm to confirm the turbidity. The minimum inhibitory concentration (MIC) was determined as the lowest concentration that inhibited visible bacterial growth.

### Checkerboard Assay

The checkerboard assay was conducted as previously described (61). The bacterial solution was prepared as described in the broth microdilution method. The antibiotic of interest was serially diluted two-fold along the x-axis, and the adjuvant is serially diluted two-fold along the y-axis in a 96-well plate. Equal volumes of bacterial solution were added to the designated wells. The first row and columns are used to verify the MIC of the antibiotic and adjuvant, respectively. The well containing only broth served as negative control, while the well containing the broth and bacterial solution served as positive control. The plate was then incubated at 37° C. for 18 hours. After incubation, Molecular Devices (United States) Emax Plus microplate reader was used to measure the OD at a wavelength of 590 nm. Synergy was determined by at least a 4-fold reduction of the MIC of both agents.

### Time-Kill Kinetics Assay

Time-kill curve analyses were performed as previously described (32). An inoculum of the culture was then diluted in 0.85% saline solution to attain 0.5 McFarland turbidity. The inoculum was further diluted in lysogeny broth (LB) containing different antibiotic and adjuvant concentrations. The culture tubes were incubated at 37° C., shaking at 250 rpm. At specific time intervals, an aliquot was taken from each tube, serially diluted in sterile phosphate-buffered saline (PBS) and plated on LB agar plates. The plates were incubated at 37° C. overnight and the bacterial colonies were counted the subsequent day. The bactericidal or bacteriostatic activity was determined based on the decrease in CFU over the designated time period. Bactericidal activity corresponded to ≥3 log reduction in CFU/mL, while bacteriostatic activity corresponded to <3 log reduction in CFU/mL.

### NPN Uptake Assay

Overnight grown P. aeruginosa PAO1 was subcultured (1 in 100) in fresh LB broth and grown to a mid-logarithmic phase (OD₆₀₀=0.4-0.6) for approximately 3.5 h. The bacterial solution was then centrifuged for 10 min at 1,200×g at room temperature to pellet the cells. Afterwards, the supernatant was decanted, and the cells were washed and resuspended in half volume of 5 mM HEPES (pH 7.2) supplemented with 5 mM glucose. On a black 96-well plate, 100 μL of cell suspension was added per well followed by the addition of 50 μL of NPN (10 μM final concentration) in 5 mM HEPES supplemented with 5 mM glucose and 5 μM CCCP. The plate was then incubated for 30 min at room temperature in darkness. After incubation, the NPN fluorescence was measured continuously (every 30 s) on a SpectraMax M2 microplate reader (Molecular Devices, USA) at an excitation wavelength of 350 nm and an emission wavelength of 420 nm. After the initial few reads, the plate was taken out of the reader and 50 μL of compound at varying concentrations were added into each well. The plate was immediately put back in the reader and the NPN fluorescence was continually measured. Cells with NPN and OM permeabilizer PMBN served as a positive control, while cells alone or NPN with compounds but no cells were used as negative controls. Three replicates were conducted, and the data were corrected for any background fluorescence.

### Cell Viability Assay

The cytotoxicity assay was conducted as previously described (24, 30). HEK293 cells were cultured in flasks with Dulbecco's Modified Eagle's Medium (DMEM) supplemented with 10% fetal bovine serum. The cells were incubated at 5% CO₂ in a humidified atmosphere at 37° C. 50 μL of media containing approximately 8000 cells were added to designated wells in a 96-well plate. The wells containing only media with no cells served as blanks. The plate was incubated for 24 h. A volume of 50 μL of doubled the final desired concentrations of the agent were subsequently added to the experimental and blank wells. After 48 h incubation, PrestoBlue reagent from Invitrogen (United States) was added to a final concentration of 10% (v/v) and plates were incubated for an additional 1 hour at 5% CO₂. Fluorescence was measured with excitation and emission wavelengths of 560 and 590 nm, respectively, using the Molecular Devices (United States) SpectraMax M2 plate reader. Values from the blank wells were subtracted from the corresponding wells with cells. The cell viability relative to the controls with vehicle was calculated. The plots indicate the mean±standard deviation of two experiments with five samples each.

The scope of the claims should not be limited by the preferred embodiments set forth in the examples but should be given the broadest interpretation consistent with the description as a whole.

### REFERENCES

(1) Tacconelli, E.; Carrara, E.; Savoldi, A.; Harbarth, S.; Mendelson, M.; Monnet, D. L.; Pulcini, C.; Kahlmeter, G.; Kluytmans, J.; Carmeli, Y.; Ouellette, M.; Outterson, K.; Patel, J.; Cavaleri, M.; Cox, E. M.; Houchens, C. R.; Grayson, M. L.; Hansen, P.; Singh, N.; Theuretzbacher, U.; Magrini, N.; Aboderin, A. O.; AI-Abri, S. S.; Awang Jalil, N.; Benzonana, N.; Bhattacharya, S.; Brink, A. J.; Burkert, F. R.; Cars, O.; Cornaglia, G.; Dyar, O. J.; Friedrich, A. W.; Gales, A. C.; Gandra, S.; Giske, C. G.; Goff, D. A.; Goossens, H.; Gottlieb, T.; Guzman Blanco, M.; Hryniewicz, W.; Kattula, D.; Jinks, T.; Kanj, S. S.; Kerr, L.; Kieny, M. P.; Kim, Y. S.; Kozlov, R. S.; Labarca, J.; Laxminarayan, R.; Leder, K.; Leibovici, L.; Levy-Hara, G.; Littman, J.; Malhotra-Kumar, S.; Manchanda, V.; Moja, L.; Ndoye, B.; Pan, A.; Paterson, D. L.; Paul, M.; Qiu, H.; Ramon-Pardo, P.; Rodríguez-Baño, J.; Sanguinetti, M.; Sengupta, S.; Sharland, M.; Si-Mehand, M.; Silver, L. L.; Song, W.; Steinbakk, M.; Thomsen, J.; Thwaites, G. E.; van der Meer, J. W.; Van Kinh, N.; Vega, S.; Villegas, M. V.; Wechsler-Fördös, A.; Wertheim, H. F. L.; Wesangula, E.; Woodford, N.; Yilmaz, F. O.; Zorzet, A. Discovery, Research, and Development of New Antibiotics: The WHO Priority List of Antibiotic-Resistant Bacteria and Tuberculosis. Lancet Infect. Dis. 2018, 18 (3), 318-327. https//doi.org/10.1016/S1473-3099(17)30753-3.
(2) Qin, S.; Xiao, W.; Zhou, C.; Pu, Q.; Deng, X.; Lan, L.; Liang, H.; Song, X.; Wu, M. Pseudomonas aeruginosa: Pathogenesis, Virulence Factors, Antibiotic Resistance, Interaction with Host, Technology Advances and Emerging Therapeutics. Signal Transduct. Target. Ther. 2022, 7 (1), 199. https//doi.org/10.1038/s41392-022-01056-1.
(3) Langendonk, R. F.; Neill, D. R.; Fothergill, J. L. The Building Blocks of Antimicrobial Resistance in Pseudomonas aeruginosa: Implications for Current Resistance-Breaking Therapies. Front. Cell. Infect. Microbiol. 2021, 11 (April), 1-22. https//doi.org/10.3389/fcimb.2021.665759.
(4) Gellatly, S. L.; Hancock, R. E. W. Pseudomonas aeruginosa: New Insights into Pathogenesis and Host Defenses. Pathog. Dis. 2013, 67 (3), 159-173. https//doi.org/10.1111/2049-632X.12033.
(5) Melander, R. J.; Melander, C. The Challenge of Overcoming Antibiotic Resistance: An Adjuvant Approach? ACS Infect. Dis. 2017, 3 (8), 559-563. https//doi.org/10.1021/acsinfecdis.7b00071.
(6) Douafer, H.; Andrieu, V.; Phanstiel, O.; Brunel, J. M. Antibiotic Adjuvants: Make Antibiotics Great Again! J. Med. Chem. 2019, 62 (19), 8665-8681. https//doi.org/10.1021/acs.jmedchem.8b01781.
(7) Domalaon, R.; Idowu, T.; Zhanel, G. G.; Schweizer, F. Antibiotic Hybrids: The next Generation of Agents and Adjuvants against Gram-Negative Pathogens? Clin. Microbiol. Rev. 2018, 31 (2), 1-45. https//doi.org/10.1128/CMR.00077-17.
(8) Tehrani, K. H. M. E.; Martin, N. I. β-Lactam/β-Lactamase Inhibitor Combinations: An Update. Medchemcomm 2018, 9 (9), 1439-1456. https//doi.org/10.1039/C8MD00342D.
(9) Dafna, Y.; G., G. C.; Alise, G.; Henrietta, A.; H., T. V.; Leonard, L. New β-Lactam-β-Lactamase Inhibitor Combinations. Clin. Microbiol. Rev. 2020, 34 (1), e00115-20. https//doi.org/10.1128/CMR.00115-20.
(10) Mauri, C.; Maraolo, A. E.; Di Bella, S.; Luzzaro, F.; Principe, L. The Revival of Aztreonam in Combination with Avibactam against Metallo-β-Lactamase-Producing Gram-Negatives: A Systematic Review of In Vitro Studies and Clinical Cases. Antibiotics. 2021. https//doi.org/10.3390/antibiotics10081012.
(11) Comely, O. A.; Cisneros, J. M.; Torre-Cisneros, J.; Rodríguez-Hernández, M. J.; Tallón-Aguilar, L.; Calbo, E.; Horcajada, J. P.; Queckenberg, C.; Zettelmeyer, U.; Arenz, D.; Rosso-Fernández, C. M.; Jiménez-Jorge, S.; Turner, G.; Raber, S.; O'Brien, S.; Luckey, A. Pharmacokinetics and Safety of Aztreonam/Avibactam for the Treatment of Complicated Intra-Abdominal Infections in Hospitalized Adults: Results from the REJUVENATE Study. J. Antimicrob. Chemother. 2020, 75 (3), 618-627. https//doi.org/10.1093/jac/dkz497.
(12) Bassetti, M.; Vena, A.; Croxatto, A.; Righi, E.; Guery, B. How to Manage Pseudomonas aeruginosa Infections. Drugs Context 2018, 7, 1-18. https//doi.org/10.7573/dic.212527.
(13) Burgess, D. S.; Hastings, R. W. Activity of Piperacillin/Tazobactam in Combination with Amikacin, Ciprofloxacin, and Trovafloxacin against Pseudomonas aeruginosa by Time-Kill. Diagn. Microbiol. Infect. Dis. 2000, 38 (1), 37-41. https//doi.org/https-//doi.org/10.1016/S0732-8893(00)00162-0.
(14) Galani, I.; Papoutsaki, V.; Karantani, I.; Karaiskos, I.; Galani, L.; Adamou, P.; Deliolanis, I.; Kodonaki, A.; Papadogeorgaki, E.; Markopoulou, M.; Maraki, S.; Damala, M.; Prifti, E.; Vagiakou, E.; Petinaki, E.; Fountoulis, K.; Tsiplakou, S.; Kirikou, H.; Souli, M.; Antoniadou, A.; Giamarellou, H. In Vitro Activity of Ceftolozane/Tazobactam Alone and in Combination with Amikacin against MDR/XDR Pseudomonas aeruginosa Isolates from Greece. J. Antimicrob. Chemother. 2020, 75 (8), 2164-2172. https//doi.org/10.1093/jac/dkaa160.
(15) Noel, A. R.; Bowker, K. E.; Attwood, M.; MacGowan, A. P. Antibacterial Effect of Ceftolozane/Tazobactam in Combination with Amikacin against Aerobic Gram-Negative Bacilli Studied in an in Vitro Pharmacokinetic Model of Infection. J. Antimicrob. Chemother. 2018, 73 (9), 2411-2417. https//doi.org/10.1093/jac/dky225.
(16) Asempa, T. E.; Nicolau, D. P.; Kuti, J. L. In Vitro Activity of Imipenem-Relebactam Alone or in Combination with Amikacin or Colistin against Pseudomonas aeruginosa. Antimicrob. Agents Chemother. 2019, 63 (9), e00997-19. https//doi.org/10.1128/AAC.00997-19.
(17) Joshi, M.; Bernstein, J.; Solomkin, J.; Wester, B. A.; Kuye, O.; Group, P. N. P. S. Piperacillin/Tazobactam plus Tobramycin versus Ceftazidime plus Tobramycin for the Treatment of Patients with Nosocomial Lower Respiratory Tract Infection. J. Antimicrob. Chemother. 1999, 43 (3), 389-397. https//doi.org/10.1093/jac/43.3.389.
(18) Mataraci Kara, E.; Yilmaz, M.; Istanbullu Tosun, A.; Özbek Çelik, B. Synergistic Activities of Ceftazidime-Avibactam in Combination with Different Antibiotics against Colistin-Nonsusceptible Clinical Strains of Pseudomonas aeruginosa. Infect. Dis. (Auckl). 2020, 52 (9), 616-624. https//doi.org/10.1080/23744235.2020.1767803.
(19) Kamrul, I.; B., S. F.; C., W. S.; J., B. M.; Saiyuri, N.; Hayoung, W.; M., Z. H.; Abu, C. M.; Tahmina, S.; H., H. Z.; A., H. P. N.; S., F. M.; A., R. J. Pharmacodynamics of Piperacillin-Tazobactam/Amikacin Combination versus Meropenem against Extended-Spectrum β-Lactamase-Producing Escherichia coli in a Hollow Fiber Infection Model. Antimicrob. Agents Chemother. 2022, 66 (9), e00162-22. https//doi.org/10.1128/aac.00162-22.
(20) Tait, J. R.; Bilal, H.; Rogers, K. E.; Lang, Y.; Kim, T.-H.; Zhou, J.; Wallis, S. C.; Bulitta, J. B.; Kirkpatrick, C. M. J.; Paterson, D. L.; Lipman, J.; Bergen, P. J.; Roberts, J. A.; Nation, R. L.; Landersdorfer, C. B. Effect of Different Piperacillin-Tazobactam Dosage Regimens on Synergy of the Combination with Tobramycin against Pseudomonas aeruginosa for the Pharmacokinetics of Critically Ill Patients in a Dynamic Infection Model. Antibiotics. 2022. https//doi.org/10.3390/antibioticsl 1010101.
(21) Idowu, T.; Ammeter, D.; Arthur, G.; Zhanel, G. G.; Schweizer, F. Potentiation of β-Lactam Antibiotics and β-Lactam/β-Lactamase Inhibitor Combinations against MDR and XDR Pseudomonas aeruginosa Using Non-Ribosomal Tobramycin-Cyclam Conjugates. J. Antimicrob. Chemother. 2019, 74 (9), 2640-2648. https//doi.org/10.1093/jac/dkz228.
(22) Ammeter, D.; Idowu, T.; Zhanel, G. G.; Schweizer, F. Development of a Nebramine-Cyclam Conjugate as an Antibacterial Adjuvant to Potentiate β-Lactam Antibiotics against Multidrug-Resistant P. aeruginosa. J. Antibiot. (Tokyo). 2019, 72 (11), 816-826. https//doi.org/10.1038/s41429-019-0221-9.
(23) Idowu, T.; Ammeter, D.; Brizuela, M.; Jackson, G.; Alam, S.; Schweizer, F. Overcoming β-Lactam Resistance in Pseudomonas aeruginosa Using Non-Canonical Tobramycin-Based Antibiotic Adjuvants. Bioorg. Med. Chem. Lett. 2020, 30 (21), 127575. https//doi.org/https//doi.org/10.1016/j.bmcl.2020.127575.
(24) Ramirez, D. M.; Ramirez, D.; Arthur, G.; Zhanel, G.; Schweizer, F. Guanidinylated Polymyxins as Outer Membrane Permeabilizers Capable of Potentiating Rifampicin, Erythromycin, Ceftazidime and Aztreonam against Gram-Negative Bacteria. Antibiotics. 2022. https//doi.org/10.3390/antibioticsl 1101277.
(25) Berry, L.; Brizuela, M.; Jackson, G.; Schweizer, F. A Niclosamide-Tobramycin Hybrid Adjuvant Potentiates Cefiderocol against P. aeruginosa. RSC Med. Chem. 2021, 12(9), 1565-1573. https//doi.org/10.1039/D1MD00206F.
(26) Temilolu, I.; G., Z. G.; Frank, S. A Dimer, but Not Monomer, of Tobramycin Potentiates Ceftolozane against Multidrug-Resistant and Extensively Drug-Resistant Pseudomonas aeruginosa and Delays Resistance Development. Antimicrob. Agents Chemother. 2020, 64 (3), e02055-19. https//doi.org/10.1128/AAC.02055-19.
(27) Hancock, R. E.; Farmer, S. W.; Li, Z. S.; Poole, K. Interaction of Aminoglycosides with the Outer Membranes and Purified Lipopolysaccharide and OmpF Porin of Escherichia coli. Antimicrob. Agents Chemother. 1991, 35 (7), 1309 LP—1314. https//doi.org/10.1128/AAC.35.7.1309.
(28) Krause, K. M.; Serio, A. W.; Kane, T. R.; Connolly, L. E. Aminoglycosides: An Overview. Cold Spring Harb. Perspect. Med. 2016, 6 (6), a027029. https//doi.org/10.1101/cshperspect.a027029.
(29) Sautrey, G.; Zimmermann, L.; Deleu, M.; Delbar, A.; Machado, L. S.; Jeannot, K.; Van Bambeke, F.; Buyck, J. M.; Decout, J.-L.; Mingeot-Leclercq, M.-P. New Amphiphilic Neamine Derivatives Active against Resistant Pseudomonas aeruginosa and Their Interactions with Lipopolysaccharides. Antimicrob. Agents Chemother. 2014, 58 (8), 4420-4430. https//doi.org/10.1128/AAC.02536-13.
(30) Idowu, T.; Arthur, G.; Zhanel, G. G.; Schweizer, F. Heterodimeric Rifampicin-Tobramycin Conjugates Break Intrinsic Resistance of Pseudomonas aeruginosa to Doxycycline and Chloramphenicol in Vitro and in a Galleria mellonella in Vivo Model. Eur. J. Med. Chem. 2019, 174, 16-32. https//doi.org/10.1016/j.ejmech.2019.04.034.
(31) Guchhait, G.; Altieri, A.; Gorityala, B.; Yang, X.; Findlay, B.; Zhanel, G. G.; Mookherjee, N.; Schweizer, F. Amphiphilic Tobramycins with Immunomodulatory Properties. Angew. Chemie—Int. Ed. 2015, 54 (21), 6278-6282. https//doi.org/10.1002/anie.201500598.
(32) Ramirez, D.; Berry, L.; Domalaon, R.; Brizuela, M.; Schweizer, F. Dilipid Ultrashort Tetrabasic Peptidomimetics Potentiate Novobiocin and Rifampicin against Multidrug-Resistant Gram-Negative Bacteria. ACS Infect. Dis. 2020, 6 (6), 1413-1426. https//doi.org/10.1021/acsinfecdis.0c00017.
(33) Wenzel, M.; Steup, J.; Ohto, K.; Weigand, J. J. Recent Advances in Guanidinium Salt Based Receptors and Functionalized Materials for the Recognition of Anions. Chem. Lett. 2021, 51 (1), 20-29. https//doi.org/10.1246/cl.210527.
(34) Lyu, Y.; Domalaon, R.; Yang, X.; Schweizer, F. Amphiphilic Lysine Conjugated to Tobramycin Synergizes Legacy Antibiotics against Wild-Type and Multidrug-Resistant Pseudomonas aeruginosa. Pept. Sci. 2019, 111 (1). https//doi.org/10.1002/bip.23091.
(35) Idowu, T.; Arthur, G.; Zhanel, G. G.; Schweizer, F. Heterodimeric Rifampicin-Tobramycin Conjugates Break Intrinsic Resistance of Pseudomonas aeruginosa to Doxycycline and Chloramphenicol in Vitro and in a Galleria mellonella in Vivo Model. Eur. J. Med. Chem. 2019, 174, 16-32. https//doi.org/10.1016/j.ejmech.2019.04.034.
(36) Baker, T. J.; Luedtke, N. W.; Tor, Y.; Goodman, M. Synthesis and Anti-HIV Activity of Guanidinoglycosides. J. Org. Chem. 2000, 65 (26), 9054-9058. https//doi.org/10.1021/jo001142e.
(37) Clinical and Laboratory Standards Institute. Performance Standards for Antimicrobial Susceptibility Testing, CLSI Supplement M100, 31st ed.; Clinical and Laboratory Standards Institute: Wayne, NJ, USA, 2021.
(38) Fair, R. J.; Hensler, M. E.; Thienphrapa, W.; Dam, Q. N.; Nizet, V.; Tor, Y. Selectively Guanidinylated Aminoglycosides as Antibiotics. Chem Med Chem 2012, 7 (7), 1237-1244. https//doi.org/https//doi.org/10.1002/cmdc.201200150.
(39) G, den H. J.; Horrevorts, A. M.; L, van G. M.; Verbrugh, H. A.; Mouton, J. W. Synergism between Tobramycin and Ceftazidime against a Resistant Pseudomonas aeruginosa Strain, Tested in an in Vitro Pharmacokinetic Model. Antimicrob. Agents Chemother. 1997, 41 (1), 95-100. https//doi.org/10.1128/AAC.41.1.95.
(40) Wang, J.; Kutter, J. P.; Mu, H.; Moodley, A.; Yang, M. Synergistic Antibacterial Effect of Inhaled Aztreonam and Tobramycin Fixed Dose Combination to Combat Multidrug-Resistant Gram-Negative Bacteria. Int. J. Pharm. 2020, 590, 119877. https//doi.org/https//doi.org/10.1016/j.ijpharm.2020.119877.
(41) Rajbharan, Y.; E., R. K.; J., B. P.; B., B. J.; J., K. C. M.; C., W. S.; L., P. D.; L., N. R.; Jeffrey, L.; A., R. J.; B., L. C. Optimization and Evaluation of Piperacillin-Tobramycin Combination Dosage Regimens against Pseudomonas aeruginosa for Patients with Altered Pharmacokinetics via the Hollow-Fiber Infection Model and Mechanism-Based Modeling. Antimicrob. Agents Chemother. 2018, 62 (5), e00078-18. https//doi.org/10.1128/AAC.00078-18.
(42) Hajira, B.; J., B. P.; Hwan, K. T.; Eun, C. S.; Y., P. A.; Antonio, O.; L., N. R.; B., L. C. Synergistic Meropenem-Tobramycin Combination Dosage Regimens against Clinical Hypermutable Pseudomonas aeruginosa at Simulated Epithelial Lining Fluid Concentrations in a Dynamic Biofilm Model. Antimicrob. Agents Chemother. 2019, 63 (11), e01293-19. https//doi.org/10.1128/AAC.01293-19.
(43) Tait, J. R.; Bilal, H.; Kim, T. H.; Oh, A.; Peleg, A. Y.; Boyce, J. D.; Oliver, A.; Bergen, P. J.; Nation, R. L.; Landersdorfer, C. B. Pharmacodynamics of Ceftazidime plus Tobramycin Combination Dosage Regimens against Hypermutable Pseudomonas aeruginosa Isolates at Simulated Epithelial Lining Fluid Concentrations in a Dynamic in Vitro Infection Model. J. Glob. Antimicrob. Resist. 2021, 26, 55-63. https//doi.org/https//doi.org/10.1016/j.jgar.2021.04.021.
(44) Bush, K.; Bradford, P. A. β-Lactams and β-Lactamase Inhibitors: An Overview. Cold Spring Harb. Perspect. Med. 2016, 6 (8), a025247. https//doi.org/10.1101/cshperspect.a025247.
(45) Perry, C. M.; Markham, A. Piperacillin/Tazobactam. Drugs 1999, 57 (5), 805-843. https//doi.org/10.2165/00003495-199957050-00017.
(46) Karen, B. Past and Present Perspectives on β-Lactamases. Antimicrob. Agents Chemother. 2018, 62 (10), e01076-18. https//doi.org/10.1128/AAC.01076-18.
(47) Helander, I. M.; Mattila-Sandholm, T. Fluorometric Assessment of Gram-Negative Bacterial Permeabilization. J. Appl. Microbiol. 2000, 88 (2), 213-219. https//doi.org/10.1046/j.1365-2672.2000.00971.x.
(48) Rothstein, D. M. Rifamycins, Alone and in Combination. Cold Spring Harb. Perspect. Med. 2016, 6 (7), a027011. https//doi.org/10.1101/cshperspect.a027011.
(49) Zgurskaya, H. I.; López, C. A.; Gnanakaran, S. Permeability Barrier of Gram-Negative Cell Envelopes and Approaches To Bypass It. ACS Infect. Dis. 2015, 1 (11), 512-522. https//doi.org/10.1021/acsinfecdis.5b00097.
(50) Jiang, M.; Karasawa, T.; Steyger, P. S. Aminoglycoside-Induced Cochleotoxicity: A Review. Front. Cell. Neurosci. 2017, 11 (October), 1-14. https//doi.org/10.3389/fncel.2017.00308.
(51) Mingeot-Leclercq, M.-P.; Tulkens, P. M. Aminoglycosides: Nephrotoxicity. Antimicrob. Agents Chemother. 1999, 43 (5), 1003 LP—1012. https//doi.org/10.1128/AAC.43.5.1003.
(52) Dagil, R.; O'Shea, C.; Nykjaer, A.; Bonvin, A. M. J. J.; Kragelund, B. B. Gentamicin Binds to the Megalin Receptor as a Competitive Inhibitor Using the Common Ligand Binding Motif of Complement Type Repeats Insight from the Nmr Structure of the Wth Complement Type Repeat Domain Alone and in Complex with Gentamicin. J. Biol. Chem. 2013, 288 (6), 4424-4435. https//doi.org/10.1074jbc.M112.434159.
(53) Moestrup, S. K.; Cui, S.; Vorum, H.; Bregengård, C.; Bjørn, S. E.; Norris, K.; Gliemann, J.; Christensen, E. I. Evidence That Epithelial Glycoprotein 330/Megalin Mediates Uptake of Polybasic Drugs. J. Clin. Invest. 1995, 96 (3), 1404-1413. https//doi.org/10.1172/JCI118176.
(54) Lopez-Novoa, J. M.; Quiros, Y.; Vicente, L.; Morales, A. I.; Lopez-Hernandez, F. J. New Insights into the Mechanism of Aminoglycoside Nephrotoxicity: An Integrative Point of View. Kidney Int. 2011, 79 (1), 33-45. https//doi.org/https//doi.org/10.1038/ki.2010.337.
(55) Findlay, B.; Zhanel, G. G.; Schweizer, F. Cationic Amphiphiles, a New Generation of Antimicrobials Inspired by the Natural Antimicrobial Peptide Scaffold. Antimicrob. Agents Chemother. 2010, 54 (10), 4049-4058. https//doi.org/10.1128/AAC.00530-10.
(56) Vaara, M. Polymyxin Derivatives That Sensitize Gram-Negative Bacteria to Other Antibiotics. Molecules 2019, 24 (2). https//doi.org/10.3390/molecules24020249.
(57) Gallardo-Godoy, A.; Muldoon, C.; Becker, B.; Elliott, A. G.; Lash, L. H.; Huang, J. X.; Butler, M. S.; Pelingon, R.; Kavanagh, A. M.; Ramu, S.; Phetsang, W.; Blaskovich, M. A. T.; Cooper, M. A. Activity and Predicted Nephrotoxicity of Synthetic Antibiotics Based on Polymyxin B. J. Med. Chem. 2016, 59 (3), 1068-1077. https//doi.org/10.1021/acs.jmedchem.5b01593.
(58) Krezdorn, J.; Adams, S.; Coote, P. J. A Galleria mellonella Infection Model Reveals Double and Triple Antibiotic Combination Therapies with Enhanced Efficacy versus a Multidrug-Resistant Strain of Pseudomonas aeruginosa. J. Med. Microbiol. 2014, 63 (PART 7), 945-955. https//doi.org/10.1099/jmm.0.074245-0.
(59) Zhanel, G. G.; DeCorby, M.; Laing, N.; Weshnoweski, B.; Vashisht, R.; Tailor, F.; Nichol, K. A.; Wierzbowski, A.; Baudry, P. J.; Karlowsky, J. A.; Lagacé-Wiens, P.; Walkty, A.; McCracken, M.; Mulvey, M. R.; Johnson, J.; Hoban, D. J. Antimicrobial-Resistant Pathogens in Intensive Care Units in Canada: Results of the Canadian National Intensive Care Unit (CAN-ICU) Study, 2005-2006. Antimicrob. Agents Chemother. 2008, 52 (4), 1430-1437. https//doi.org/10.1128/AAC.01538-07.
(60) Hoban, D. J.; Zhanel, G. G. Introduction to the CANWARD Study (2007-11). J. Antimicrob. Chemother. 2013, 68 (suppl_1), i3-i5. https//doi.org/10.1093/jac/dkt021.
(61) Lyu, Y.; Yang, X.; Goswami, S.; Gorityala, B. K.; Idowu, T.; Domalaon, R.; Zhanel, G. G.; Shan, A.; Schweizer, F. Amphiphilic Tobramycin-Lysine Conjugates Sensitize Multidrug Resistant Gram-Negative Bacteria to Rifampicin and Minocycline. J. Med. Chem. 2017, 60 (9), 3684-3702. https//doi.org/10.1021/acs.jmedchem.6b01742.

1. A method of treating a Pseudomonas aeruginosa infection comprising:
administering an effective amount of a compound having a formula as set forth below for compound 5 or compound 8 to an individual who has or is suspected of having a Pseudomonas aeruginosa infection

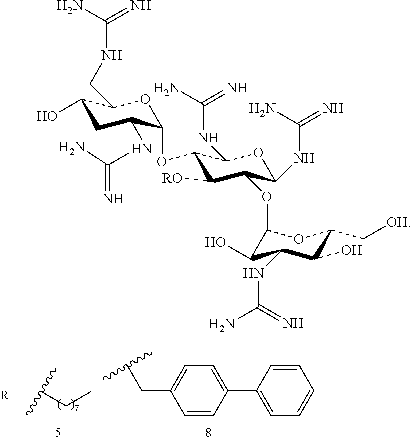

2. The method according to claim 1 wherein the Pseudomonas aeruginosa infection is caused by a multi-drug resistant Pseudomonas aeruginosa strain.

3. The method according to claim 1 wherein the compound is administered in combination with at least one antibiotic.

4. The method according to claim 3 wherein the antibiotic is a β-lactam/β-lactamase antibiotic.

5. The method according to claim 4 wherein the β-lactam/β-lactamase antibiotic is at least one β-lactam/β-lactamase antibiotic selected from the group consisting of ceftazidime, aztreonam, avibactam, piperacillin and meropenem.

6. The method according to claim 3 wherein the antibiotic is rifampicin.

7. The method according to claim 3 wherein the compound is administered with ceftazidime and avibactam.

8. A method of permeabilizing a Gram Negative bacteria outer membrane comprising:
administering an effective amount of a compound having a formula as set forth below for compound 5 or compound 8 to a Gram Negative bacteria

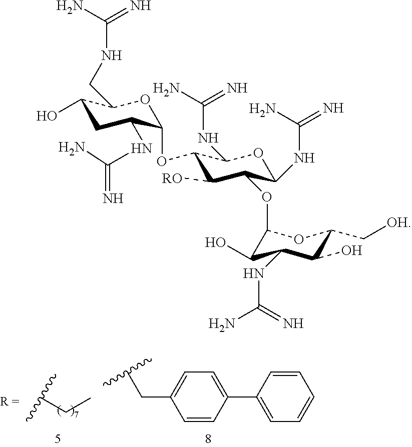

9. The method according to claim 8 wherein the Gram Negative bacteria is Pseudomonas aeruginosa.

10. A compound having a formula as set forth below for compound 5 or compound 8 for permeabilizing a Gram Negative bacteria outer membrane [chemical structure]
